(19) United States
(12) Patent Application Publication
(10) Pub. No.: US 2008/0010728 A1
(43) Pub. Date: Jan. 17, 2008

(54) Pair of goggles

(76) Inventor: Daryl F. Speed, Vancouver (CA)

Correspondence Address: FREDERICK KAUFMAN, SUITE 403, 1937 PENDRELL STREET, VANCOUVER, BC, V6G 1T4, CA

(21) Appl. No.: 11/895,587

(22) Filed: Aug. 27, 2007

(51) Int. Cl. A61F 9/02

(52) U.S. Cl. 2/426

(57) ABSTRACT

A pair of goggles including two housing units and two eye protecting units, each of the latter being secured within one of the former units that incorporate a flexible, convex, lateral extension for adjusting to a temple area. Each eye protecting units incorporates a cup-shaped structure and is provided, at its back edge, by a convex wall of reduced size that expands backwards into a deflectable lateral extension, the former and the latter being contiguous with a gasket for sealing. The adjustment of the flexible, lateral extension does not interfere with the deflectable, lateral extension and with the sealing unit. The latter has its cantilever length relatively longer than the deflectable, lateral extension, so that during and after stretching of the adjustable unit for strapping—the flexible, lateral extension forms a zone, wherein the deflectable, lateral extension is confined and spaced from the former.

### I. BACKGROUND OF THE INVENTION

1. Definition of the Invention

The present invention relates, in general, to swimming equipment, and, in particular, to a pair of goggles for this type of sport.

2. Description of the Prior Art

The prior art describes many types and shapes of goggles. In general, common to all these goggles is the fact that the forces, which pull back the sides of the pair of goggles, can negatively affect the comfortableness for the wearer's eye sockets, and, even, sometimes, related to the latter, the adequacy of the water-tightness.

For example, U.S. Pat. No. 7,194,771 granted on Mar. 27, 2007 to Chou for a “FRAME/LENS COMBINATION FOR SWIMMING GOGGLES” descries and illustrates a combination which includes a rigid frame with stretching resistance and a rigid lens also with stretching resistance. The frame is annular and includes an opening having an extension that tapers outward to form a tapered end section. A perimeter wall delimiting the opening includes an outer section that delimits the tapered end section. The outer section is engaged with an associated end of a head strap. The goggles disclosed in the foregoing patent has a main shortcoming which resides in the fact that by stretching the head strap the rigid frame impinges directly on the adjacent rigid lens which deforms the eye socket sealing beyond a comfortable condition that can also cause an inadequate water-tightness. Another example is U.S. Pat. No. 6,317,897 granted on Nov. 20, 2001 to Chiang for “SWIMMING GOGGLES”. This patent discloses a pair of goggles comprising a binding frame including left and right frame units, left and right lens frames, a nose bridge, left and right assembling devices, and a headband device. In the left and right frame units, each receives a lens frame, and each lens frame receives a lens. In the left and right frame units of the binding frame, each includes at least one clasp unit and one check unit. Each lens frame comprises at least one clasp device corresponding in position to the clasp unit on the left and right frame units. Each assembling device is received in a corresponding assembling hole on the left and right frame units, such that the clasp units engage the check units through the assembling devices. Thus, the right and left lens frames are securely fastened in the left and right frame units of the binding frame. The aforementioned swimming goggles have the same shortcoming as the previous one. Yet another example is U.S. Pat. No. 6,029,284 granted on Feb. 29, 2000 to Kawashima et al. for “SWIMMING GOGGLES”. This patent discloses a peripheral wall of a lens member that is gradually thickened rearward from a front lens so that the peripheral wall has a height extending beyond a top of a flange of the lens member and a first groove extending along the flange is formed. A second groove of a cushion pad is engaged with an entire periphery of the flange so that the pad is exposed at an outer end of the lens member. The main drawback of the disclosed swimming goggles consists in the fact that the pair of arms, especially in the zone of their vertical holes, situated at the end of the latter and intended for the insertion of a head band, faces the back end edges of the lens member. During and after tightening of the head band, the ends of the pair of arms abut against the back end edges rendering uncomfortable the use of the swimming goggles.

### II. SUMMARY OF THE INVENTION

What are therefore needed are goggles which are designed to adhere closely to the eyes, temples and occipital back area, without essentially affecting the comfortableness and the water-tightness.

Thus, a first objective of the present invention is to design a pair of swimming goggles wherein the stretching or tightening of an adjustable attaching strap is not detrimental to the comfortableness and to an adequate sealing.

A second objective of the present invention is to design a pair of swimming goggles which incorporates appropriate surfaces that permit a laminar flow of the encountered water.

Broadly stating, the pair of goggles according to the present invention comprises, in combination,
two housing units symmetrically disposed;
two eye protecting units, each one of the latter, adaptable to form an ocular chamber in front of a wearer's eye, are surroundably engaged by and securably attached within one of the two housing units with which it is associated;
a sealing unit, directly attached to each one of the two eye protecting units and adaptable, due to its basically flexible construction, to provide a sealingly fittable contact with a contour of an eye socket, and, thereby, to cause a reliable water proof effect; and
an adjustable unit for strapping.

Each of said two housing units incorporates in its monobloc, transparent structure a flexible, lateral extension, adaptable for adjusting positionally to an eye temple area by allowing a deflection capable to sustain a flexural stress and to elastically return to the initial position. A convexity of the flexible, lateral extension provides spring properties, which, in addition, is adjustable by an appropriate selection of a hardness of a plastic material used for the two housing units, geometry of the convexity and a thickness of the plastic material.

Each of said two means for eye protecting incorporate a cup-shaped structure made of the same material as each of said two means for housing and have at its back edge a convex wall of a relatively reduced size that expands backwards into a deflectable lateral extension, the former and the latter being contiguous with said means for sealing.

Each of the two housing units, permanently engaged with one of the two eye protecting units with which is associated and having the flexible, lateral extension provided with such suitable convexity and cantilever length—the latter being relatively longer than the deflectable, lateral extension—that during and after stretching of the adjustable unit for strapping, the flexible, lateral extension forms a free zone, wherein the deflectable, lateral extension is confined and spaced from the former.

The flexible, lateral extension being also shaped and sized that its length and convexity shields any protuberances and/or other features of the pair of goggles situated in the eye temple junction and/or temple zones, from producing a hydrodynamic resistance.

The stretching of the pair of goggles basically causes an adjustment of the flexible, lateral extension, without essentially interfering with the deflectable, lateral extension and with the sealing unit—the latter being already adapted to be seated on eye sockets—except in the case of extreme final adjustments.

In another broad aspect of the present invention, for use in a pair of goggles, intended especially for swimming, the pair of goggles including
two housing units symmetrically disposed;
two eye protecting units, each one of the latter, adaptable to form an ocular chamber in front of a wearer's eye, are surroundably engaged by and securably attached within one of the two housing units with which it is associated;
a sealing unit, directly attached to each one of the two eye protecting units and adaptable, due to its basically flexible construction, to provide a sealingly fittable contact with a contour of an eye socket, and, thereby, to cause a reliable water proof effect; and
an adjustable unit for strapping.

Each of said two housing units incorporates in its monobloc, transparent structure a flexible, lateral extension, adaptable for adjusting positionally to an eye temple area by allowing a deflection capable to sustain a flexural stress and to elastically return to the initial position. A convexity of the flexible, lateral extension provides spring properties, which, in addition, is adjustable by an appropriate selection of a hardness of a plastic material used for the two housing units, geometry of the convexity and a thickness of the plastic material.

Each of said two means for eye protecting incorporates a cup-shaped structure made of the same material as each of said two means for housing and has at its back edge a convex wall of a relatively reduced size that expands backwards into a deflectable lateral extension, the former and the latter being contiguous with said means for sealing.

Each of the two housing units, permanently engaged with one of the two eye protecting units with which is associated and having the flexible, lateral extension provided with such suitable convexity and cantilever length—the latter being relatively longer than the deflectable, lateral extension—that during and after stretching of the adjustable unit for strapping the flexible, lateral extension forms a free zone, wherein the deflectable, lateral extension is confined and spaced from the former.

The flexible, lateral extension being also shaped and sized that its length and convexity shields any protuberances and/or other features of the pair of goggles situated in the eye temple junction and/or temple zones, from producing a hydrodynamic resistance.

The stretching of the pair of goggles basically causes an adjustment of the flexible, lateral extension, without essentially interfering with the deflectable, lateral extension and with the sealing unit—the latter being already adapted to be seated on eye sockets—except in the case of extreme final adjustments.

### III. BRIEF DESCRIPTION OF THE DRAWINGS

The subject matter of the invention is particularly pointed out and distinctively claimed in the concluding portion of the specification. The invention, however, both in structure and operation may be better understood by reference to the following description taken in conjunction with the subjoined claims and the accompanying drawings of which

FIG. 1 illustrates a front elevation view of the pair of goggles according to the present invention;

FIG. 2 is a longitudinal cross section taken along line A-A of FIG. 1;

FIG. 3 illustrates the right half of the first elevation view shown in FIG. 1;

FIG. 4 is a longitudinal cross section taken along line B-B of FIG. 3;

FIG. 4′ shows a fragment of FIG. 4 without the nose bridge and the gasket;

FIG. 5 is an enlarged view of detail C shown on FIG. 4; 0

FIG. 6 is an enlarged view of detail D shown of FIG. 4; and

FIG. 7 is a fragmentary view of the gasket.

### IV. DESCRIPTION OF THE PREFERRED EMBODIMENTS

Before the variants of the present invention are explained in the forthcoming description, it is to be understood that the invention is not limited in its application to the details of construction and the arrangements of components set forth in the following description or illustrated in the accompanying drawings. The invention is capable of other embodiments and of be practiced or of being carried out in various ways. Also, it is to be understood that the terms used herein with reference to the orientation of a pair of goggles (such as, for example, terms like “forwards”, “backwards”, “laterally” and the like), are only used to better understand the description of the present invention, and do not alone indicate or imply that the invention and its use, referred to, must have a particular position.

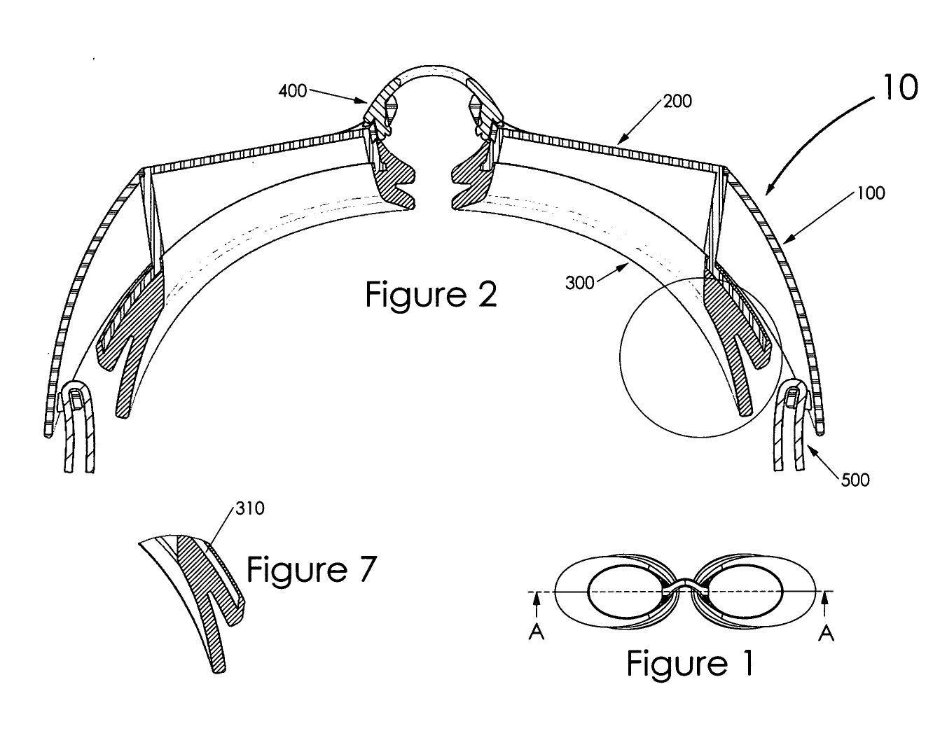

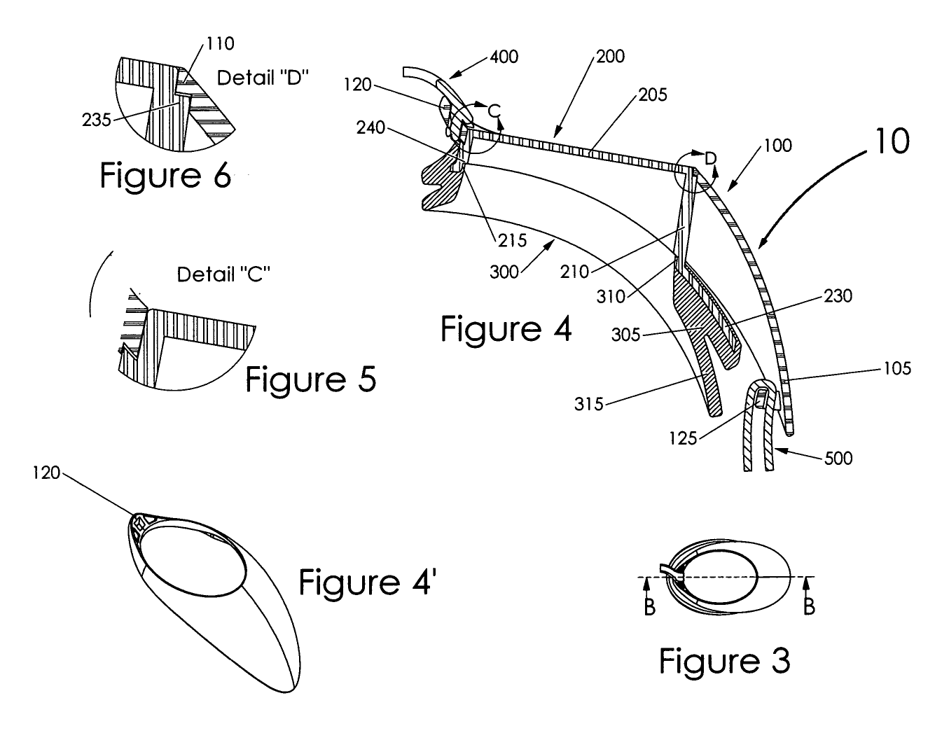

Generally, a pair of goggles 10, according to the present invention, comprises (see FIGS. 1-4)
two housing frames 100, laterally symmetric;
two eye protective cup lenses 200, each one of the latter forming an ocular chamber in front of a wearer's eye, is surroundably engaged by and securably attached within an associate housing frame 100;
a gasket 300 directly attached to each eye protective cup lens 200 and adaptable, due to its basically flexible construction, to provide a sealingly fittable contact with the contour of the eye socket of the wearer, and, thereby, to cause a reliable waterproof effect;
a nose bridge 400 connecting the two housing frames 100 between inner ends thereof situated symmetrically adjacent the nose of the wearer; and
an adjustable, locking-unlocking strap 500, extending between outer ends of the housing frames 100, which outer ends are opposite to the above mentioned inner ends, adjustable, locking-unlocking strap 500 being made usually of stretchable material such that it can follow, and fit to, both the contours of the wearer's eye temple and occipital zones.

Describing now in detail, each housing frame 100 is made of plastic material having a relatively high tensile strength and, generally, includes a monobloc, convex (as viewed from outside), rigid and transparent structure. Housing frame 100 includes a flexible, lateral extension 105, adaptable for better adjusting positionally to the eye temple area by allowing a deflection capable to sustain a flexural stress and to elastically return to the initial position. The convexity of flexible, lateral extension 105 provides very good spring properties, which, in addition, can be easily adjusted by an appropriate selection of the hardness of the plastic material, the geometry of the convexity and, obviously, the thickness of the plastic material forming housing frame 100, namely its convex structure.

Each housing frame 100 incorporates frontally a backwardly facing rabbet 110 that circumscribes peripherally an oval opening, geometrically defined by an intersection of housing frame 100 with a vertical plane.

Housing frame 100 further incorporates, at its inner end (front extremity), an orifice provided protrusion 120 extending outwardly and forwardly and intended for attaching to nose bridge 400.

Flexible, lateral extension 105 of housing frame 100 incorporates, as well, proximately to its outer end, an inside U-shaped attaching member 125 that is so positionally located that the laminar flow of water across the pair of goggles in this zone is not disturbed.

Each one of the two eye protective cup lenses 200 is made of the same material as housing frame 100 and include, generally, a monobloc, rigid and transparent structure. The latter includes a flat portion 205, oval shaped and frontally disposed with respect to the rest of the structure. A continuous wall 210 extends backwardly from flat portion 205 and is slightly inclined outwardly. Continuous wall 210 is formed by a generatrix that gradually increases its length along the periphery of flat portion 205, with its minimum situated in the proximity of orifice provided protrusion 120 and reaching its maximum length-at the opposite end.

Continuous wall 210 is limited by an oval-shaped, back edge 215 that is surrounded by a convex wall 220 of relatively reduced size. The latter extends backwards and laterally from oval-shaped, back edge 215 into a relatively larger deflectable lateral extension 230. Flat portion 205 incorporates, as well, a forwardly facing rabbet 235 that circumscribes its periphery.

Oval shaped, back edge 215 is provided with a shoulder 240. The use of the latter will be described later in the present disclosure.

Backwardly facing rabbet 110 of housing frame 100 is so shaped and sized as to engage forwardly facing rabbet 235 of flat portion 205, respectively of an associate eye protective cup lens 200.

Backwardly and forwardly facing rabbets 110 and 230 are generally bonded together by gluing or by ultrasound welding. Any other suitable fitting means such as, for example, snapping can be equally used.

Optionally, besides the oval shape in the aforementioned structures, other surfaces at least partially defined by a continuous curve can be conveniently used.

Alternatively, flat portion 205 can be replaced by a convex surface (as viewed from outside).

Gasket 300 has an accordion-type median zone 305 and is formed frontally along its entire length with a groove 310 configured to snugly engage shoulder 240 of oval-shaped back edge 215 and deflectable lateral extension 230.

Gasket 300 ends backwardly with a flange 315 which, due to flexibility and softness of the chosen material, deforms, when in use, to comply with contours of the eye sockets, thereby preventing ingress of water into an eye protective cup lens 200.

Gasket 300 is usually joined to eye protective cup lens 200 by gluing or by two components injection molding.

Besides adjustable, locking-unlocking strap 500, other conventional types of straps can be used.

Each housing frame 100 being firmly engaged with an associate eye protective cup lens 200, its flexible, lateral extension 105, due to its convexity and its length, which is relatively longer than deflectable, lateral extension 230, forms a free zone F wherein the latter is confined and spaced from the former (when adjustable, locking-unlocking strap 500 is stretched).

Supplementarily, flexible, lateral extension 105 has its length and convexity so conveniently chosen that it shields any protuberances and/or other features of the pair of goggles 10, situated in the eye temple junction and/or temple zones, from producing a hydrodynamic resistance (for example, part of the gasket, strap connection etc.).

When during stretching, a tension load is applied to flexible, lateral extension 105, the latter is subjected to a bending moment which translates into its better adjustment to the wearer's temple.

Flexible, lateral extension 105 has a cantilever structure, but is able to sustain flexural stress and return to its initial state, mainly due to its reinforced point of contraflexure where no bending moment occurs. The latter point is situated in a zone where backwardly facing rabbet 110 of housing frame 100 is bonded with forwardly facing rabbet 230 of flat portion 205 of an associate eye protective cup lens 200.

From the foregoing description one can directly infer that the tightening of the pair of goggles 10 basically causes an adjustment of flexible, lateral extension 105, without essentially interfering with deflectable, lateral extension 230 of eye protective lenses 200, and with gaskets 300, the latter being already seated on eye sockets, except perhaps in the case of extreme final adjustments. If this will have happened, it will further deform the gaskets which can be uncomfortable for the wearer of the pair of goggles 10. Further tightening will deform the gaskets 300 which become uncomfortable for the wearer and can affect negatively the water-tightness.

What I claim is:

1. A pair of goggles intended especially for swimming, comprising in combination
two means of housing symmetrically disposed;
two means for eye protecting, each one of the latter, adaptable to form an ocular chamber in front of a wearer's eye, being surroundably engaged by and securably attached within one of said two means of housing with which it is associated;
means for sealing, directly attached to each one of said two means for eye protecting and adaptable, due to its basically flexible construction, to provide a sealingly fittable contact with a contour of an eye socket of the wearer's eye, and, thereby, to cause a reliable water proof effect; and
adjustable means for strapping;
each of said two means for housing incorporating a monobloc, transparent structure with a flexible, lateral extension, adaptable for adjusting positionally to an eye temple area by allowing a deflection capable to sustain a flexural stress and to elastically return to the initial position, a convexity of said flexible, lateral extension providing spring properties, which, in addition, are adjustable by an appropriate selection of a hardness of a plastic material used for said means for housing, geometry of said convexity and a thickness of said plastic material;
each of said two means for eye protecting incorporating essentially a cup-shaped structure, generally made of the same material as each of said two means for housing and having at its back edge a convex wall of a relatively reduced size that expands backwards into a deflectable lateral extension, the former and the latter being contiguous with said means for sealing;
each of said two means for housing, permanently engaged with one of said two means for eye protecting with which is associated and having said flexible, lateral extension provided with such suitable convexity and cantilever length—the latter being relatively longer than said deflectable, lateral extension—that during and after stretching of said adjustable means for strapping, said flexible, lateral extension forms a free zone, wherein said deflectable, lateral extension is confined and spaced from the former;
said flexible, lateral extension being also shaped and sized that its length and convexity shields any protuberances and/or other features of said pair of goggles situated in the eye temple junction and/or temple zones, from producing a hydrodynamic resistance;
whereby,
said stretching of said pair of goggles, being basically adaptable to cause an adjustment of said flexible, lateral extension, without essentially interfering with said deflectable, lateral extension and with said means of sealing—the latter being already seated on eye sockets—except in the case of extreme final adjustments.

2. For use in a pair of goggles, intended especially for swimming, and including
two means of housing symmetrically disposed;
two means for eye protecting, each one of the latter, adaptable to form an ocular chamber in front of a wearer's eye, being surroundably engaged by and securably attached within one of said two means of housing with which it is associated;
means for sealing, directly attached to each one of said two means for eye protecting and adaptable, due to its basically flexible construction, to provide a sealingly fittable contact with a contour of an eye socket of the wearer's eye, and, thereby, to cause a reliable water proof effect; and
adjustable means for strapping;
each of said two means for housing incorporating in its monobloc, transparent structure a flexible, lateral extension, adaptable for adjusting positionally to an eye temple area by allowing a deflection capable to sustain a flexural stress and to elastically return to the initial position, a convexity of said flexible, lateral extension providing spring properties, which, in addition, are adjustable by an appropriate selection of a hardness of a plastic material used for said means for housing, geometry of said convexity and a thickness of said plastic material;
each of said two means for eye protecting incorporating a cup-shaped structure, made of the same material as each of said two means for housing and having at its back edge a convex wall of a relatively reduced size that expands backwards into a deflectable lateral extension, the former and the latter being contiguous with said means for sealing;
each of said two means for housing, permanently engaged with one of said two means for eye protecting with which is associated and having said flexible, lateral extension provided with such suitable convexity and cantilever length—the latter being relatively longer than said deflectable, lateral extension—that during and after stretching of said adjustable means for strapping, said flexible, lateral extension forms a free zone, wherein said deflectable, lateral extension is confined and spaced from the former;
said flexible, lateral extension being also shaped and sized that its length and convexity shields any protuberances and/or other features of said pair of goggles situated in the eye temple junction and/or temple zones, from producing a hydrodynamic resistance;
whereby,
said stretching of said pair of goggles basically being adaptable to cause an adjustment of said flexible, lateral extension, without essentially interfering with said deflectable, lateral extension and with said means of sealing—the latter being already seated on eye sockets—except in the case of extreme final adjustments.
